(19) United States
(12) Patent Application Publication
(10) Pub. No.: US 2024/0207501 A1
(43) Pub. Date: Jun. 27, 2024

(54) STABILIZING INTRA-OCULAR PRESSURE AT ONSET OF ASPIRATION

(71) Applicant: JOHNSON & JOHNSON SURGICAL VISION, INC., Irvine, CA (US)

(72) Inventors: Vadim Gliner, Haifa (IL); Assaf Govari, Haifa (IL)

(21) Appl. No.: 18/089,407

(22) Filed: Dec. 27, 2022

(51) Int. Cl. A61M 1/00; A61F 9/007

(52) U.S. Cl. CPC A61M 1/74; A61F 9/00745; A61M 1/774; A61B 2217/005; A61B 2217/007; A61M 2205/3334; A61M 2205/3341

(57) ABSTRACT

A pumping system for a phacoemulsification system, consisting of: a phacoemulsification probe, an irrigation tube providing irrigation fluid into an eye, an irrigation pump pumping the irrigation fluid, an aspiration tube conveying aspiration fluid from the eye, an aspiration pump pumping the aspiration fluid from the eye, an aspiration pump sensor providing a first signal indicating activation of the aspiration pump, and a pressure sensor providing a second signal indicating intraocular pressure (IOP) in the eye. A controller receives the first signal and second signals, and in response controls the irrigation pump to alter a registered flow rate of the irrigation fluid to a preset adjusted flow rate until a threshold, dependent on at least one of a time of operation of the irrigation pump at the preset adjusted flow rate and the second signal, is crossed. When crossed the irrigation fluid is pumped at the registered flow rate.

### FIELD OF THE DISCLOSURE

This disclosure relates generally to ocular surgery, and specifically to the value of the intra-ocular pressure (IOP) during the surgery.

### BACKGROUND

A cataract is a cloudy area in the lens of the eye that leads to a decrease in vision. Phacoemulsification is a modern cataract surgery method in which the eye's internal lens is emulsified with an ultrasonic handpiece and aspirated from the eye. The aspirated fluids may be replaced with irrigation of balanced salt solution to maintain the intra-ocular pressure (IOP) of the eye. Once the natural lens has been removed, an intraocular lens (IOL) implant may then be placed into the remaining lens capsule.

### BRIEF DESCRIPTION OF THE DRAWINGS

The present disclosure will be understood from the following detailed description, taken in conjunction with the drawings in which:

FIG. 1 is a pictorial view of a phacoemulsification apparatus constructed to operate in accordance with an example of the present disclosure;

FIG. 2 is a schematic block diagram illustrating an irrigation sub-system and an aspiration sub-system of the phacoemulsification apparatus; and

FIG. 3 is a flowchart of steps performed in operation of the phacoemulsification apparatus.

### DESCRIPTION OF EXAMPLES

### Overview

In a phacoemulsification procedure, assumed herein to be performed to remove the lens of a patient's eye, a physician uses a phacoemulsification handpiece to insert a hollow needle into the eye. The needle is vibrated ultrasonically, causing the lens to break into particles, and the particles are aspirated from the eye via an aspiration line from the needle. In order to maintain the intraocular pressure (IOP) of the eye at a target IOP value and within acceptable bounds while the aspiration occurs, the eye is separately irrigated via an irrigation line to the needle. The irrigation flow and the aspiration flow need to be balanced to maintain the target IOP level and to prevent exceeding the acceptable bounds, since breaching either an upper or a lower IOP bound may cause irreparable damage to the eye.

The IOP may be monitored with a pressure sensor embedded in the irrigation line used for the irrigation flow or otherwise positioned near the surgical site. A dedicated irrigation controller, typically including a feedback control loop e.g., a PID (proportional integral derivative) control loop, is configured to receive output from the pressure sensor and compare the output to a setpoint value stored in memory associated with the controller. The setpoint value is typically the target IOP value that is desired to be maintained during the surgical procedure. An identified difference between the output received and the setpoint value triggers the controller to change the flow rate of the pump to compensate for the difference detected and return the IOP to the desired level.

Typically, during the procedure, the site being operated on is irrigated continuously, and the aspiration is toggled as required by the physician, typically by the physician activating a foot pedal, e.g., from position 1 to position 2. The present applicant has found that when the physician activates the aspiration with the foot pedal, the activation is typically followed by an abrupt dip in the IOP. Typically, it may take 1-3 seconds for the controller with feedback loop to return the IOP to the target level desired. This delay may occur for two reasons. One reason is the delay due to the time it takes the pressure sensor to identify the dip. The other reason is due to the numerous iterations that are required to converge the IOP to the setpoint value.

Similarly, a dip in the IOP may also occur during a vacuum surge that may occur post occlusion due to vacuum buildup that occurred during the occlusion and/or based on reactivating the aspiration flow after temporarily cutting off the aspiration to avoid the vacuum surge.

However, the reduction in IOP is typically significantly larger in response to the physician toggling from irrigation only to irrigation and the aspiration flow. The larger TOP reduction, and the consequential longer time to return to a target IOP value, are problematic.

Embodiments of the disclosure solve the problem by providing additional input to the feedback control loop regarding the state of the user interface device, e.g., the foot pedal for toggling the aspiration ON/OFF. For example, the PID irrigation controller may be configured to receive an indication immediately with activation of the aspiration. Direct input from the user interface device avoids any delay in recognizing the pressure drop based on the output from the pressure sensor. Based on the input received, the flow rate of the irrigation line may be temporarily boosted to compensate for the dip in pressure. The boosted irrigation rate may be dynamically defined based on the target IOP and the current pressure reading. Optionally and additionally, on receipt of the indication, one or more parameters used to operate the controller are temporarily altered to compensate for the expected drop in IOP.

In some example embodiments, the delayed response time is further reduced by temporarily using a setpoint value that is significantly higher than the target IOP for controlling the irrigation flow. The significantly higher setpoint value initiates more aggressive response by the feedback loop and thereby reduces the response time. In some example embodiments, the temporary setpoint value may be 30%-100% higher than the target IOP, e.g., 90 mmHg-140 mmHg and may be defined based on empirical data and stored in memory associated with the controller. In some example embodiments, the temporary setpoint value is dynamically defined based on a detected offset from the target IOP and a current flow rate provided by the irrigation pump. Optionally, a lookup table or defined relationship between offset from the target IOP, a current flow rate and the temporary setpoint value is stored in memory in association with the irrigation controller. The parameter alteration temporarily provides a relatively large increase in the irrigation flow rate provided by the irrigation pump, to prevent or reduce the expected dip. The parameter alteration also ensures that the time to return to the target IOP is reduced.

In some example embodiments, additional input is also provided to the feedback control loop in response to activation of an anti-vacuum surge (AVS) device that is configured to cut-off the aspiration flow based on detecting a vacuum surge. Optionally, activation of the AVS is an indication that an abrupt dip in the IOP has occurred and this input may be used to initiate temporarily changing the setpoint value used by the feedback control loop. In some example embodiments, temporary setpoint value used in response to a vacuum surge may be other than the temporary setpoint value used based on detecting that a physician has activated aspiration. Optionally, the temporary setpoint value for a vacuum surge may be 20%-100% of the target IOP.

Optionally, more than one temporary setpoint value may be stored in memory associated with the irrigation controller. In some example embodiments, the temporary setpoint value is dynamically defined based on a detected offset from the target IOP and a current flow rate provided by the irrigation pump regardless of whether the drop in IOP is due to a vacuum surge event or toggling of the aspiration by the physician. Optionally, a lookup table or defined relationship between offset from the target IOP, a current flow rate and the temporary setpoint value is stored in memory in association with the irrigation controller.

### System Description

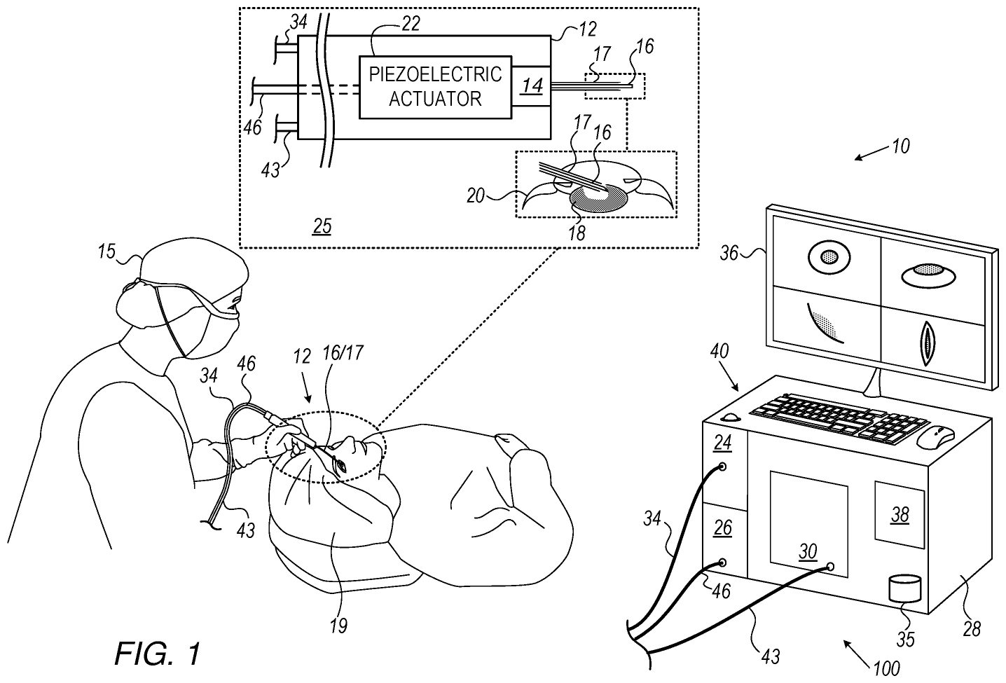

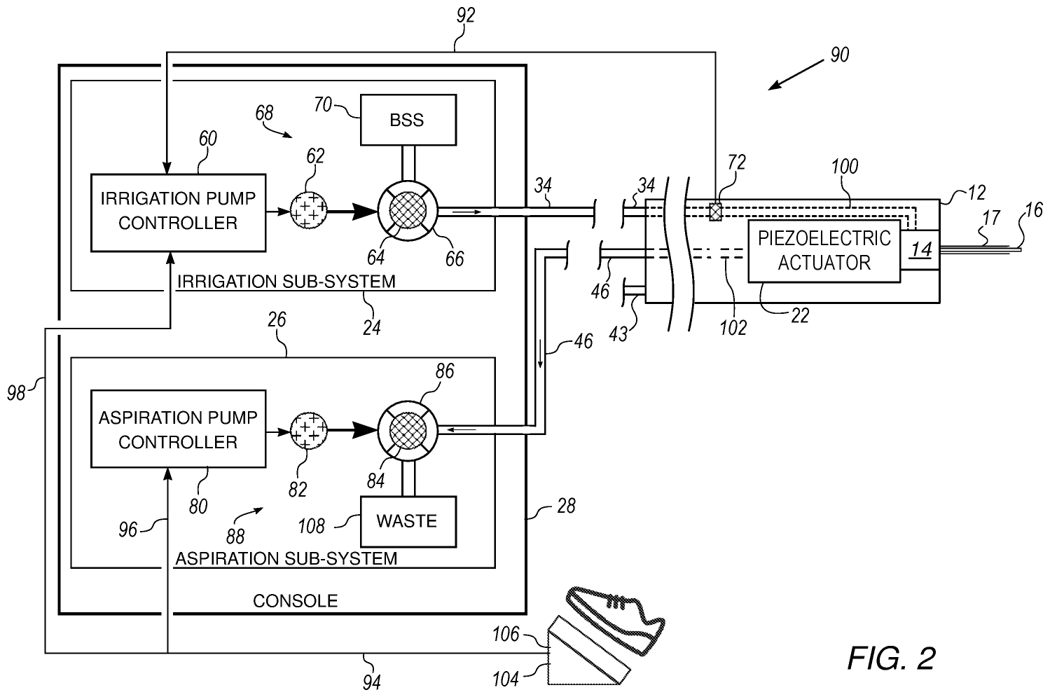

FIG. 1 is a pictorial view of a phacoemulsification apparatus 10 constructed to operate in accordance with an example of the present disclosure. FIG. 1 includes an inset 25, and as shown in the figure and the inset apparatus 10 includes a phacoemulsification probe/handpiece 12 comprising a needle 16 and a coaxial irrigation sleeve 17 that at least partially surrounds the needle 16 and that creates a fluid pathway between the external wall of the needle 16 and the internal wall of the irrigation sleeve 17. Needle 16 is configured to be inserted into a lens capsule 18 of an eye 20 of a patient 19. Needle 16 is mounted on a horn 14 of probe 12, and is shown in inset 25 as a straight needle. However, any suitable needle may be used with the phacoemulsification probe 12, for example, a curved or bent tip needle commercially available from Johnson & Johnson Surgical Vision, Inc, Santa Ana, CA, USA. A physician 15 holds handpiece 12 so as to perform a phacoemulsification procedure on the eye of patient 19. The physician may activate the handpiece using a foot pedal, described below with reference to FIG. 2.

Handpiece 12 comprises a piezoelectric actuator 22, which is configured to vibrate horn 14 and needle 16 in one or more vibration modes of the combined horn and needle. Except where otherwise stated, in the following description the actuator is assumed to receive signals so as to generate a linear vibration mode. During the phacoemulsification procedure the vibration of needle 16 is used to break a cataract into small pieces.

Elements of apparatus 10 are under overall control of a processor 38 in a console 28. Functions of processor 38 are describe in more detail below.

During the phacoemulsification procedure, an irrigation sub-system 24 in console 28 pumps irrigation fluid to irrigation sleeve 17 so as to irrigate the eye. The fluid is pumped via an irrigation tube 34, also herein termed an irrigation tubing line 34, running from the console 28 to the probe 12. An aspiration sub-system 26, also located in console 28, aspirates eye fluid and waste matter (e.g., emulsified parts of the cataract) from the patient's eye via needle 16. Aspiration sub-system 26 comprises a pump which produces a vacuum that is connected from the sub-system to probe 12 by a vacuum aspiration tubing line 46, also herein termed an aspiration line 46 or an aspiration tube 46.

The functions and structure of irrigation sub-system 24 and aspiration sub-system 26 are described with respect to FIG. 2 below.

During the procedure, it is important to maintain the intra-ocular pressure (IOP) of the eye within acceptable bounds. Operation of the two pumping systems, if not controlled, may affect the IOP adversely, and as is described herein, embodiments of the disclosure provide control for the pumping systems so that the IOP is maintained within the acceptable bounds.

FIG. 2 is a schematic block diagram illustrating irrigation sub-system 24 and aspiration sub-system 26. Irrigation sub-system 24 comprises an irrigation pump 68, herein by way of example assumed to comprise a progressive cavity pump (PCP) having an internal rotor 64 and an external stator 66. Pump 68 is driven by a motor 62, which is controlled by an irrigation pump controller 60. In an embodiment of the disclosure controller 60 is a proportional integral derivative (PID) controller.

Irrigation tubing line 34 is connected to an irrigation channel 100 incorporated in handpiece 12. Irrigation fluid, typically a balanced salt solution (BSS), is pumped, from an irrigation fluid reservoir 70, by pump 68 through tubing line 34 and irrigation channel 100 to irrigation sleeve 17. A pressure sensor 72 is located in channel 100, the sensor providing a measure of the IOP, and a connecting line 92 indicates that the signal generated by the pressure sensor is provided to controller 60.

Controller 60 uses the signal from sensor 72 as negative feedback, in a feedback loop 90, when operating motor 62, to maintain the IOP measured by the sensor substantially constant. Loop 90 comprises sensor 72, controller 60, foot-pedal 104 (described further below), and motor 62, and the loop is configured, inter alia, so that an increase in pressure measured by sensor 72 leads controller 60 to reduce the irrigation flow rate, e.g., by driving motor 62 at a lower RPM (rotation per minute). Likewise, a decrease in pressure measured by sensor 72 leads controller 60 to increase the irrigation flow rate, e.g., by driving motor 62 at a higher RPM (rotation per minute). In addition, when an indication is received that the physician has activated the aspiration, e.g., with the foot pedal, controller 60 is configured to temporarily operate with a higher setpoint value of the IOP to temporarily boost a rate at which the target IOP reached.

Aspiration sub-system 26 comprises an aspiration pump 88, herein by way of example assumed to comprise a PCP having an internal rotor 84 and an external stator 86. Pump 88 is driven by a motor 82, which is controlled by an irrigation pump controller 80. In an embodiment of the disclosure controller 80 is and/or includes a PID controller. Aspiration tubing line 46 is connected to an aspiration channel 102 in handpiece 12. When operative, pump 88 aspirates matter acquired by needle 16, via channel 102 and tubing 46, to a waste matter container 108.

During the procedure, physician 15 operates the irrigation and aspiration sub-systems, using a sub-system user interface. In an embodiment of the disclosure a foot-pedal 104 acts as the sub-system user interface, and in a disclosed embodiment the foot-pedal has three positions: a first position where the irrigation sub-system alone is activated, and a second position where both the irrigation and the aspiration sub-system are activated and a third position where the irrigation sub-system, the aspiration sub-system and the ultrasound sub-system are activated. In the second position foot-pedal 104 provides a signal for the aspiration sub-system, so that in this position the foot-pedal acts as an aspiration sub-system sensor 106.

While for simplicity signal lines 94, 96, and 98 are shown as being connected directly between foot-pedal 104 and controllers 60 and 80, in practice signals from the foot-pedal may be relayed to, and/or adjusted for, the controllers by processor 38. Similarly, signals from pressure sensor 72 may be relayed to, and/or adjusted for, irrigation pump controller 60 by processor 38.

Optionally, irrigation and/or aspiration may be operated with other types of pumps.

Returning to FIG. 1, irrigation sub-system 24 and aspiration sub-system 26 are both under overall control of processor 38. The processor provides input to controller 60 to control the rate at which the irrigation sub-system pumps its fluid. The processor also provides input to controller 80 to control the vacuum pressure produced by the aspiration sub-system when the system is activated.

Some or all of the functions of processor 38 may be combined in a single physical component or, alternatively, implemented using multiple physical components. The physical components may comprise hard-wired or programmable devices, or a combination of the two. In some examples, at least some of the functions of processor 38 may be carried out by suitable software stored in a memory 35. The software may be downloaded to a device in electronic form, over a network, for example. Alternatively, or additionally, the software may be stored in tangible, non-transitory computer-readable storage media, such as optical, magnetic, or electronic memory.

Processor 38 may receive user-based commands via a system user interface 40, which may include setting and/or adjusting a vibration mode and/or a frequency of piezoelectric actuator 22, setting and/or adjusting a stroke amplitude of needle 16, and setting and/or adjusting a default irrigation rate and a default aspiration rate of irrigation sub-system 24 and aspiration sub-system 26. Additionally, or alternatively, processor 38 may receive user-based commands from controls located in handpiece 12, to, for example, select a trajectory for needle 16.

Processor 38 may present results of the phacoemulsification procedure on a display 36. In an example, user interface 40 and display 36 may be one and the same, such as a touch screen graphical user interface.

The procedure illustrated in FIG. 1 may include further elements, which are omitted for clarity of presentation. For example, physician 15 typically performs the procedure using a stereo-microscope or magnifying glasses, neither of which are shown. Physician 15 may use other surgical tools, in addition to probe 12, which are also not shown to maintain clarity and simplicity.

Console 28 comprises a piezoelectric drive module 30, which is coupled to piezoelectric actuator 22, via processor 38, using electrical wiring running in a cable 43.

As stated above, pump 68 may be PCP and the flow rate and pressure provided by the pump is defined by RPM (revolutions per minute) of the motor. The pressure Pᵣₚₘ delivered by the pump is given by equation (1):

$${P}_{r p m} = β \cdot {I}_{r p m} \qquad ( 1 )$$

where β is a constant.

$$Δ P ( t ) = {P}_{s e t} - {P}_{a c t} ( t ) \qquad ( 2 )$$

where Pₛₑₜ is target IOP, e.g., 70 mmHg, and P_{act}(t) is the pressure measured by pressure sensor 72 at time t.

In an embodiment of the disclosure, the value of output Iᵣₚₘ(t) by PID controller 60 is given by equation (3):

$${I}_{r p m} ( t ) = {K}_{c} [ Δ P ( t ) + \frac{1}{{t}_{i}} {\int }_{0}^{t} Δ P ( t ) d t + {t}_{d} \frac{d Δ P ( t )}{d t} ] \qquad ( 3 )$$

where K_{c}, tᵢ, t_{d} are respective coefficients of the proportional, integral, and differential terms of the controller, and ΔP(t) is given by equation (2).

In some example embodiments, when a drop in the IOP is predicted based on controller 60 receiving input from a user interface device indicating that the aspiration flow has been initiated, or based on controller 60 receiving input from an AVS device, a new setpoint value to the feedback loop is temporarily set for controlling the irrigation flow. The value of Pₛₑₜ used by irrigation controller 60 in equation (3), is temporarily altered to a new setpoint value P_{newset}. The value of P_{newset} may be dynamically defined by equation (45):

$${P}_{n e w s e t} = β \cdot {I}_{r p m} + α \cdot {( {P}_{s e t} - {P}_{a c t} ( t ) )}^{n} \qquad ( 4 )$$

where α and n are constants. Optionally and typically, n=1.

In another example embodiment P_{newset} may be alternatively be defined based on equation

$${P}_{n e w s e t} = {P}_{s e t} + α \cdot {( {P}_{s e t} - {P}_{a c t} ( t ) )}^{n} \qquad ( 5 )$$

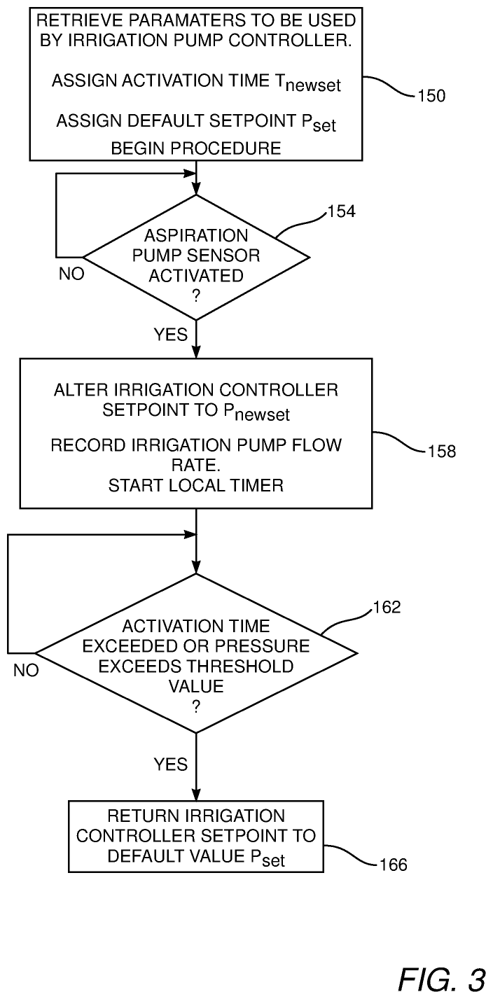

FIG. 3 is a flowchart of steps performed in operation of phacoemulsification apparatus 10 illustrated in FIG. 1. The flowchart illustrates the application of equation (6).

In an initial step 150, values of constants used by equations (4) or (5) are stored in memory 35, and are retrieved from the memory as necessary by processor 38 and irrigation pump controller 60.

In addition, an activation time T_{newset}, during which the new setpoint P_{newset} is to be used, and a value of Pₛₑₜ, the default pressure setpoint, are stored and retrieved from memory 35.

In an embodiment T_{newset} is assigned a value of 2 s, Pₛₑₜ is assigned a value of 70 mmHg, and α and n are assigned values so that Pₛₑₜ increases by approximately 50% so that P_{newset} is approximately 100 mmHg. Optionally, P_{newset} increases by 30%-100%. Optionally, T_{newset} is dynamically defined based on Pₛₑₜ−P_{act}(t). For example, once Pₛₑₜ−P_{act}(t) falls below a defined threshold, the setpoint value used returns to Pₛₑₜ instead of P_{newset}.

Once the parameters above are retrieved, the procedure may be begun.

As the procedure is being performed, processor 38 continuously monitors signals from foot-pedal 104, i.e. the signal generated by sensor 106 when the foot-pedal activates the aspiration sub-system by being positioned into its second position.

The continuous monitoring of the foot-pedal is indicated by a first condition step 154, wherein the activation sub-system is monitored, and continues while the condition returns negative, indicating the sub-system has not been activated.

When condition step 154 returns positive, indicating that the aspiration sub-system 26 has been activated, control proceeds to a controller alteration step 158.

In step 158 processor 38 calculates a value of P_{newset} according to equation (4) or (5) by finding and recording the value of P_{act}(t_{act}) at an activation time t_{act} when condition step 154 begins to return positive. The value of P_{newset} is applied to irrigation pump controller 60, so that the controller operates according to equation (3).

In addition, at activation time t_{act} processor 38 starts a local timer. As explained below, the local timer is used to evaluate the time of operation using P_{newset}.

It will be appreciated that applying the value of P_{newset} to controller 60 alters the flow rate value output by the controller, and thus the irrigation fluid flow rate produced by pump 68. Typically, the alteration comprises an increased irrigation fluid flow rate. It will also be appreciated that the increased irrigation fluid flow rate achieved by applying the value of P_{newset} also accelerates a speed of response of pump 68.

Control from step 158 continues to a second condition 162, where processor 38 determines when the change initiated by first condition returning positive is to be halted. To make this determination, processor 38 compares the local timer value with T_{newset}. In addition, processor 38 compares the value measured by pressure sensor 72 with the value of Pact(t) recorded in step 158.

The two comparisons act as thresholds, so that if the local timer value exceeds T_{newset}, the system is assumed to have crossed a first threshold. If the pressure measured by pressure sensor 72 is within a preset fraction of P_{act}(t_{act}), the system is assumed to have crossed a second threshold. In an embodiment the preset fraction is approximately 90%.

In condition step 162 processor monitors both comparisons. If neither threshold is crossed the condition returns negative. If either threshold is crossed, the condition returns positive, and control continues to a final step 166.

In final step 166, processor 38 returns the irrigation setpoint value used by controller 60 to the values of initial step 150, so that the controller operates using equation (3).

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

### EXAMPLES

Example 1. A pumping system for a phacoemulsification system (10), comprising:
a phacoemulsification probe (12) having a distal end comprising a needle (16) configured to be inserted into an eye;
an irrigation tube (34) configured to be connected to the probe and to provide irrigation fluid into the eye;
an irrigation pump (68) configured to be connected to the irrigation tube and to pump the irrigation fluid into the eye;
an aspiration tube (46) configured to be connected to the probe and to convey aspiration fluid from the eye;
an aspiration pump (88) configured to be connected to the aspiration tube and to pump the aspiration fluid from the eye;
an aspiration pump sensor (106) configured to provide a first signal indicative of activation of the aspiration pump;
a pressure sensor (72) configured to provide a second signal indicative of intraocular pressure (IOP) in the eye; and
a pump controller (60) configured to:
receive the first signal provided by the pump sensor,
receive the second signal provided by the pressure sensor,
and,
responsively to the provided first signal:
register a flow rate of the irrigation fluid;
control the irrigation pump to alter the registered flow rate of the irrigation fluid to a preset adjusted flow rate until a predefined threshold, dependent on at least one of a time of operation of the irrigation pump at the preset adjusted flow rate and the second signal, is crossed, and, on crossing of the threshold, control the irrigation pump to pump the irrigation fluid at the registered flow rate.

Example 2. The pumping system according to example 1, wherein the preset adjusted flow rate comprises an increase by a preset fraction of the registered flow rate.

Example 3. The pumping system according to example 2, wherein the preset fraction is a function of a value of the IOP recorded when the first signal is received by the pump controller.

Example 4. The pumping system according to example 2, wherein the pump controller is configured to use the first signal provided by the pump sensor to maintain the IOP at a target IOP, and wherein the preset fraction is a function of the target IOP.

Example 5. The pumping system according to example 1, wherein controlling the irrigation pump comprises accelerating a speed of response of the pump.

Example 6. The pumping system according to example 1, wherein the pump controller is configured to:
receive input from an anti-vacuum surge (AVS) device configured to block aspiration flow to a distal end of the phacoemulsification handpiece; and
temporarily boost the preset IOP provided to the feedback control loop based on the input received from the AVS device.

Example 7. A progressive cavity pump (PCP) (68) for supplying irrigation flow to a phacoemulsification system, the pump comprising:
a rotor (64) housed in a stator (66);
a motor (62) configured to rotate the rotor; and
a controller (60) configured to control a flow rate provided by the PCP to maintain a preset intraocular pressure (IOP), wherein the controller includes a feedback loop that is configured to:
receive pressure readings from a pressure sensor (72) configured to monitor IOP;
receive input from a user interface (104) configured to control activation of aspiration flow through the phacoemulsification system; and
temporarily boost the preset IOP provided to the feedback control loop based on the input from the user interface indicating actuation of the aspiration flow.

Example 8. The progressive cavity pump according to example 7, wherein the preset IOP is boosted by a predetermined pressure comprising a preset fraction of the preset IOP.

Example 9. The progressive cavity pump according to example 8, wherein the preset fraction is a function of a value of the IOP recorded when the input is received from the user interface.

Example 10. The progressive cavity pump according to example 8, wherein the preset IOP comprises a target IOP, and wherein the preset fraction is a function of the target IOP.

Example 11. The progressive cavity pump according to example 7, wherein the boosting of the preset IOP is halted if either a preset time of boosting is exceeded, or an IOP measured by the pressure sensor exceeds a preset fraction of a value of the IOP recorded when the input is received from the user interface.

Example 12. The progressive cavity pump according to example 7, wherein the controller is configured to:
receive input from an anti-vacuum surge (AVS) device configured to block aspiration flow to a distal end of the phacoemulsification handpiece; and
temporarily boost the preset IOP provided to the feedback control loop based on the input received from the AVS device.

Example 13. A method for operating a pumping system for a phacoemulsification system (10), comprising:
providing a phacoemulsification probe (12) having a distal end comprising a needle (16) configured to be inserted into an eye;
connecting an irrigation tube (34) to the probe;
connecting an irrigation pump (68) to the irrigation tube and pumping the irrigation fluid into the eye;
connecting an aspiration tube (46) to the probe;
connecting an aspiration pump (88) to the aspiration tube and pumping the aspiration fluid from the eye;
configuring an aspiration pump sensor (106) to provide a first signal indicative of activation of the aspiration pump;
configuring a pressure sensor (72) to provide a second signal indicative of intraocular pressure (IOP) in the eye;
receiving the first signal provided by the pump sensor;
receiving the second signal provided by the pressure sensor;
responsively to the first signal, registering a flow rate of the irrigation fluid; and
controlling the irrigation pump to alter the registered flow rate of the irrigation fluid to a preset adjusted flow rate until a predefined threshold, dependent on at least one of a time of operation of the irrigation pump at the preset adjusted flow rate and the second signal, is crossed, and, on crossing of the threshold, control the irrigation pump to pump the irrigation fluid at the registered flow rate.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A pumping system for a phacoemulsification system, comprising:
a phacoemulsification probe having a distal end comprising a needle configured to be inserted into an eye;
an irrigation tube configured to be connected to the probe and to provide irrigation fluid into the eye;
an irrigation pump configured to be connected to the irrigation tube and to pump the irrigation fluid into the eye;
an aspiration tube configured to be connected to the probe and to convey aspiration fluid from the eye;
an aspiration pump configured to be connected to the aspiration tube and to pump the aspiration fluid from the eye;
an aspiration pump sensor configured to provide a first signal indicative of activation of the aspiration pump;
a pressure sensor configured to provide a second signal indicative of intraocular pressure (IOP) in the eye; and
a pump controller configured to:
receive the first signal provided by the pump sensor,
receive the second signal provided by the pressure sensor,
and,
responsively to the provided first signal:
register a flow rate of the irrigation fluid;
control the irrigation pump to alter the registered flow rate of the irrigation fluid to a preset adjusted flow rate until a predefined threshold, dependent on at least one of a time of operation of the irrigation pump at the preset adjusted flow rate and the second signal, is crossed, and, on crossing of the threshold, control the irrigation pump to pump the irrigation fluid at the registered flow rate.

2. The pumping system according to claim 1, wherein the preset adjusted flow rate comprises an increase by a preset fraction of the registered flow rate.

3. The pumping system according to claim 2, wherein the preset fraction is a function of a value of the IOP recorded when the first signal is received by the pump controller.

4. The pumping system according to claim 2, wherein the pump controller is configured to use the first signal provided by the pump sensor to maintain the IOP at a target IOP, and wherein the preset fraction is a function of the target IOP.

5. The pumping system according to claim 1, wherein controlling the irrigation pump comprises accelerating a speed of response of the pump.

6. The pumping system according to claim 1, wherein the pump controller is configured to:
receive input from an anti-vacuum surge (AVS) device configured to block aspiration flow to a distal end of the phacoemulsification handpiece; and
temporarily boost the preset IOP provided to the feedback control loop based on the input received from the AVS device.

7. A progressive cavity pump (PCP) for supplying irrigation flow to a phacoemulsification system, the pump comprising:
a rotor housed in a stator;
a motor configured to rotate the rotor; and
a controller configured to control a flow rate provided by the PCP to maintain a preset intraocular pressure (IOP), wherein the controller includes a feedback loop that is configured to:
receive pressure readings from a pressure sensor configured to monitor IOP;
receive input from a user interface configured to activation flow through control of aspiration the phacoemulsification system; and
temporarily boost the preset IOP provided to the feedback control loop based on the input from the user interface indicating actuation of the aspiration flow.

8. The progressive cavity pump according to claim 7, wherein the preset IOP is boosted by a predetermined pressure comprising a preset fraction of the preset IOP.

9. The progressive cavity pump according to claim 8, wherein the preset fraction is a function of a value of the IOP recorded when the input is received from the user interface.

10. The progressive cavity pump according to claim 8, wherein the preset IOP comprises a target IOP, and wherein the preset fraction is a function of the target IOP.

11. The progressive cavity pump according to claim 7, wherein the boosting of the preset IOP is halted if either a preset time of boosting is exceeded, or an IOP measured by the pressure sensor exceeds a preset fraction of a value of the IOP recorded when the input is received from the user interface.

12. The progressive cavity pump according to claim 7, wherein the controller is configured to:
receive input from an anti-vacuum surge (AVS) device configured to block aspiration flow to a distal end of the phacoemulsification handpiece; and
temporarily boost the preset IOP provided to the feedback control loop based on the input received from the AVS device.

13. A method for operating a pumping system for a phacoemulsification system, comprising:
providing a phacoemulsification probe having a distal end comprising a needle configured to be inserted into an eye;
connecting an irrigation tube to the probe;
connecting an irrigation pump to the irrigation tube and pumping the irrigation fluid into the eye;
connecting an aspiration tube to the probe;
connecting an aspiration pump to the aspiration tube and pumping the aspiration fluid from the eye;
configuring an aspiration pump sensor to provide a first signal indicative of activation of the aspiration pump;
configuring a pressure sensor to provide a second signal indicative of intraocular pressure (IOP) in the eye;
receiving the first signal provided by the pump sensor;
receiving the second signal provided by the pressure sensor;
responsively to the first signal, registering a flow rate of the irrigation fluid; and
controlling the irrigation pump to alter the registered flow rate of the irrigation fluid to a preset adjusted flow rate until a predefined threshold, dependent on at least one of a time of operation of the irrigation pump at the preset adjusted flow rate and the second signal, is crossed, and, on crossing of the threshold, control the irrigation pump to pump the irrigation fluid at the registered flow rate.

14. The method according to claim 13, wherein the preset adjusted flow rate comprises an increase by a preset fraction of the registered flow rate.

15. The method according to claim 14, wherein the preset fraction is a function of a value of the IOP recorded when the first signal is received by the pump controller.

16. The method according to claim 14, and comprising using the first signal provided by the pump sensor to maintain the IOP at a target IOP, and wherein the preset fraction is a function of the target IOP.

17. The method according to claim 13, wherein controlling the irrigation pump comprises accelerating a speed of response of the pump.
